(19) United States
(12) Patent Application Publication
(10) Pub. No.: US 2006/0167563 A1
(43) Pub. Date: Jul. 27, 2006

(54) Sub-malleolar non-articulating prosthetic foot with improved dorsiflexion

(76) Inventors: Christopher L. Johnson, Mountain Park, OK (US); Aaron Taszreak, China Township, MI (US)

Correspondence Address: GIFFORD, KRASS, GROH, SPRINKLE & CITKOWSKI, P.C, PO BOX 7021, TROY, MI, 48007-7021, US

(21) Appl. No.: 11/338,995

(22) Filed: Jan. 25, 2006

Related U.S. Application Data
(60) Provisional application No. 60/646,670, filed on Jan. 25, 2005.

(51) Int. Cl. A61F 2/66

(52) U.S. Cl. 623/52; 623/55

(57) ABSTRACT

A prosthetic foot includes a sole plate having a body formed of resilient material. The sole plate is elongated along an anterior and posterior axis wherein an anterior portion of the plate defines a toe portion and the posterior portion defines a heel portion. An ankle member having a planar portion in contact with the sole plate is rigidly affixed to the sole plate at the heel portion. The ankle member has an extension portion positioned anterior of the planar portion and separated from the planar portion by a transition portion. A resilient pad is disposed between the contact portion of the ankle member and the sole plate. The resilient pad and extension portion of ankle member define a gap such that as a user's weight is transferred anterior following initial contact, the extension portion of the ankle member tilts forward reducing the gap formed between the resilient pad and the extension portion for improving the dorsiflexion of the prosthetic foot.

### RELATED APPLICATION

This application claims priority of U.S. Provisional Patent Application Ser. No. 60/646,670 filed Jan. 25, 2005, which is incorporated herein by reference.

### FIELD OF THE INVENTION

This invention relates generally to foot prostheses. More specifically, the invention relates to sub-malleolar, non-articulating foot prostheses.

### BACKGROUND OF THE INVENTION

A prosthetic foot is a very important component of leg prostheses. A prosthetic foot must reliably store and release energy while flexing in a number of degrees of motion so as to properly coordinate with the muscular action when a user is walking, running or standing in place. In addition, a prosthetic foot must provide a reliable action over a large number of operational cycles under ambient conditions which include exposure to dust, dirt, various liquids and a large range of operational temperatures. In addition, it is generally desirable that a prosthetic foot be relatively low in cost and easy to maintain.

The art has made very large advances in producing prosthetic feet which imitate natural foot action. Many of these devices are mechanically complex and employ a number of moving parts. While such devices provide extremely good and reliable performance characteristics, their cost and complexity limits their use, particularly in high volume applications and in user communities which do not have a sophisticated technical infrastructure to support and maintain such devices.

Sub-malleolar (all mechanical parts are located below the bony projections or maleoli at the top of the ankle joint), non-articulating devices generally comprise mechanically simple prosthetic devices which include a non-articulating ankle member used in combination with a sole plate. The sole plates of such devices are usually formed of a relatively rigid and durable material, such as carbon fiber or the like. The use of rigid materials allow for a longer service life of the sole plate which is exposed to large force loads. However, such rigid materials can lead to problems in that the sole plate does not have sufficient flexibility to allow for a natural gait. Very rigid materials or thicknesses of materials may promote durability but do not flex to allow the heel of the sole plate to maintain contact with the ground surface a sufficient period of time to approximate a natural foot action. Such premature heel rise leads to a less natural gait of a user of the prosthetic device, with increased mental and physical fatigue.

There is therefore a need in the art for a prosthetic device that has a more flexible sole plate allowing for improved dorsiflexion improving the gait of a user while simultaneously maintaining a long service life and durability.

As will be explained in detail herein below, the present invention provides a prosthetic foot which does not include any articulated members, but which emulates feet containing articulating members, providing a comfortable, natural foot action over a very long service life. In addition, the prosthetic foot of the present invention does not require any periodic maintenance or adjustment, and is relatively low in cost. These and other advantages of the invention will be apparent from the drawings, discussion and description which follow.

### SUMMARY OF THE INVENTION

A prosthetic foot includes a sole plate having a body formed of resilient material of lower material stiffness or overall part stiffness than is typical to the art. The sole plate is elongated along an anterior and posterior axis wherein an anterior portion of the plate defines a toe portion and the posterior portion defines a heel portion. An ankle member having a planar portion in contact with the sole plate is rigidly affixed to the sole plate at the heel portion. The ankle member has an extension portion positioned anterior of the planar portion and separated from the planar portion by a transition portion. A resilient pad is disposed between the contact portion of the ankle member and the sole plate. The resilient pad and extension portion of ankle member define a gap such that as a user's weight is transferred anterior following initial contact, the extension portion of the ankle member tilts forward reducing the gap formed between the resilient pad and the extension portion for improving the dorsiflexion of the prosthetic foot while simultaneously retaining the critical requirement of durability by arching the sole plate about the resilient pad, distributing sole plate internal stresses.

### BRIEF DESCRIPTION OF THE DRAWINGS

FIG. 1 is a side view of a prosthetic foot having a resilient member disposed between the sole plate and ankle in accordance with the present invention;

FIG. 2 is a side view of the prosthetic of FIG. 1 with the resilient member moved anterior in relation to the sole plate;

FIG. 3 is a side view of the ankle of the prosthetic foot of the present invention;

FIG. 4 is a side view of the prosthetic of FIG. 1 having a resilient member with a reduced thickness.

### DETAILED DESCRIPTION OF THE INVENTION

The present invention is directed to a prosthetic foot which is attachable to a leg prosthesis and which provides for a natural foot action. The prosthetic foot of the present invention includes a sole plate formed from a body of resilient material. The sole plate is elongated along an anterior and posterior axis, and the anterior portion of the sole plate defines the toe portion of the prosthetic foot and the posterior portion defines the heel portion of the prosthetic foot. An ankle member includes a planar portion that is rigidly affixed to the sole plate at the heel portion. The ankle member also includes an extension portion which is anterior of the planar portion. When the ankle member is affixed to the sole plate, the extension portion is spaced apart from the surface of the sole plate. The prosthetic foot also includes a resilient pad which is disposed in the space between the extension portion of the ankle member and the sole plate.

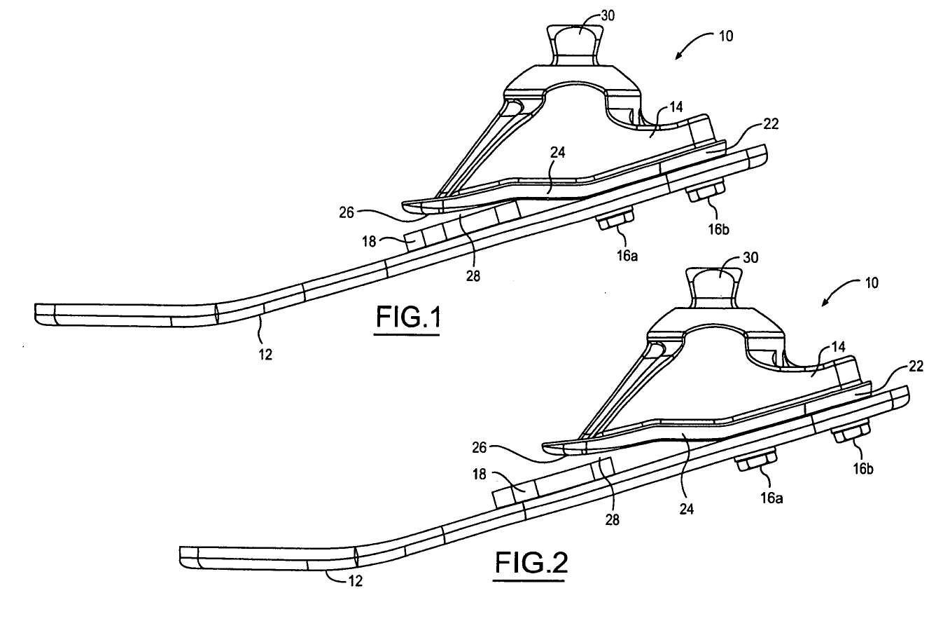

Referring now to FIG. 1, there is shown an embodiment of a prosthetic foot 10 structured in accord with the principles of the present invention. The foot 10 includes a sole plate 12. The sole plate 12 is formed from a resilient material, and within the context of this disclosure, a resilient material is understood to mean a material which may be bent or otherwise deformed by a force applied to it, and which in the absence of such force returns to its original shape. In the prior art, the resilient material of the sole plate typically has a high degree of stiffness, often more than is needed for proper gait mechanics, because of design requirements for durability. In the present invention, the sole plate 12 may be fabricated from a polymeric composite, such as a fiber-reinforced composite. Reinforcing material may be of carbon fiber, glass fiber, ceramic fibers, or any other high strength fiber. The material is not limited to anisotropic or quasi-isotropic composite materials, but can be of isotropic materials such as spring steel. In the present invention, the material utilized for the sole plate 12 may be a glass fiber reinforced polymeric composite.

The sole plate 12 generally has a shape corresponding to the sole of a foot, and in this regard is generally elongated along an anterior and posterior axis wherein the anterior portion of the plate corresponds to the toe portion of the foot, and the posterior portion corresponds to the heel portion of the foot. The thickness and composition of the sole plate 12 may vary to tune its flexing characteristics to the needs of a specific user. In the present invention the sole plate 12 may have flexibility or material modulus properties of from 4 to 19 Mpsi. The thickness of the material of the sole plate may be adjusted to maintain the flexibility in the above referenced range. For example, using a lower stiffness material the sole plate may be thicker. While using stiffer materials the sole plate may be thinner. A more flexible sole plate 12 allows for dorsiflexion of the sole plate 12 to allow the heel portion to remain in contact with the ground longer to simulate a natural gait. The flexibility properties of the sole plate 12 in conjunction with the resilient pad 18 allow for an improved gait while providing a durable prosthetic foot 10 that has a long service life.

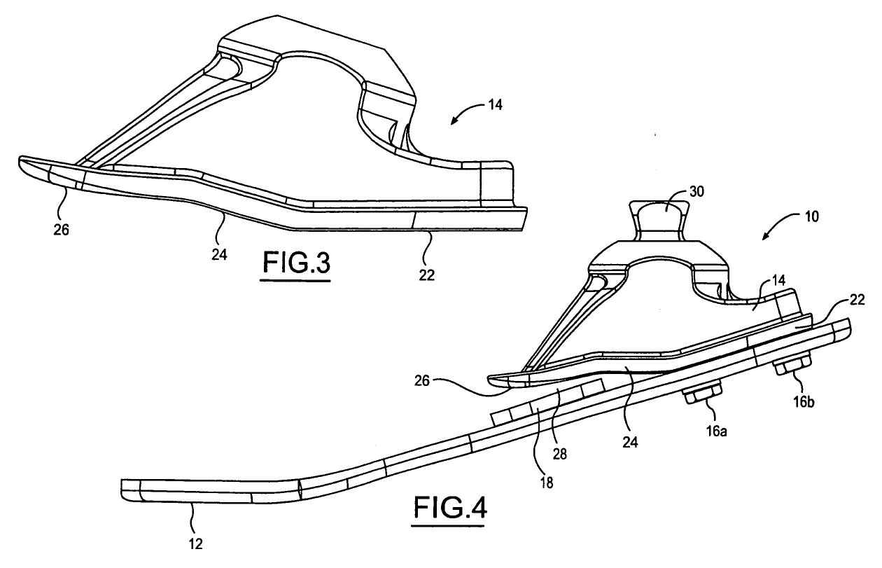

Referring to FIGS. 1 and 3, an ankle member 14 includes a bottom surface 20 that is operative to contact the sole plate 12. The bottom surface 20 includes a planar portion 22 that is in the region of the heel of the sole plate 12. The planar portion 22 angles upward at a transition portion 24 spaced anterior to the planar portion 22. Spaced anterior of the transition portion 24 is an extension portion 26 that includes a slightly curved profile angling upward.

The ankle member 14 is coupled to the sole plate 12, such that the planar 22 portion contacts the heel region of the sole plate 12. A pair of bolts 16a, 16b passes through slots formed in the ankle member 14 so as to rigidly affix the ankle member 14 to the heel portion of the sole plate 12. The extension portion 26 of the ankle member 14 is spaced apart from the subjacent portions of the sole plate 12 when the ankle assembly 14 is affixed to the sole plate 12. The extension portion 26 of the ankle member 14 and the resilient pad 18 define a gap 28. The ankle member 14 further includes a connector 30 associated therewith. This connector 30 functions to join the foot to the remainder of a leg prosthesis.

As is further shown in FIG. 1, the resilient pad 18 is disposed in the space between the extension portion 18 of the ankle assembly 14, and the subjacent portions of the sole plate 12. This resilient pad 18 is typically formed from a flexible polymeric material. The material may be selected to tune the prosthesis for an individual user. In one aspect of the present invention, the material may be a closed cell polyester or polyether foam. The foam may have a density of from 20-60 pounds per cubic foot (pcf). The material may also be a rubber, such as SBR, the durometer range of a rubber material would form 60-90 Shore A. It should be realized that other materials with varying properties may be used by the present invention to form the resilient pad 18. The resilient pad 18 may be moved anterior along the sole plate 12, as shown in FIG. 2 to affect the flexibility characteristics of the sole plate 12 to adjust the gait of a user, as will be described in more detail below. Additionally, the thickness and hardness of the resilient pad 18 may be adjusted, as shown in FIG. 4, again to adjust the flexibility of the sole plate 12.

The term gait as used in the specification can be defined as a style of walking. Gait is a highly complex activity involving the reciprocal motion of the legs, arms and trunk and can be divided into two distinct phases occurring simultaneously in opposite legs and sequentially in the same leg in a collective gait cycle. The gait cycle is divided into two phases: swing and stance. The stance phase is the period of time when the foot is in ground contact, while the swing phase refers to when the foot is not on the weight-bearing surface. The stance phase includes three stages: 1) contact, when the heel strikes the ground; 2) midstance, which begins with full ground contact and ends with heel lift and 3) propulsion, during which time the foot prepares to leave the ground.

During the contact stage of a human foot, the foot lands at the posterolateral aspect of the heel, with most of the weight on the outer edge. A gradual shifting of weight to the inner edge follows as the foot moves down and inward to a position of pronation. This is accomplished by internal rotation of the subtalar joint. The arch flattens to distribute the force of heel strike and midfoot arches unlock, relieving tension and encouraging flexibility of arch ligaments to facilitate shock absorption and adaptation to uneven terrain. The ball of the foot makes initial contact with the ground. During the midstance stage, the weight shifts from the posterior to the anterior portion of the foot. Pronation ends as the foot begins to roll upward and forward to a position of supination. The subtalar joint and midfoot structures that had relaxed become rigid, preparing the foot to act as a lever that will launch the body forward. Body weight moves directly over the foot. During propulsion, the foot effectively becomes a lever with the posterior structures providing force and the ball serving as a fulcrum. With weight shifted to the outer edge, the foot effectively moves downward and away from the leg. Toeing off brings the foot away from the ground and launches it to the swing phase, when no weight is borne until the stance phase repeats at the next ground contact.

In a prosthetic device, the sole plate in conjunction with the socket of the prosthetic device define a heel lever, the perpendicular distance from the heel of the sole plate to the center line of the socket and a toe lever, the perpendicular distance from the center line of the socket to the anterior end of the sole plate. These levers can be manipulated by moving the socket in relationship to the foot to shorten either the toe lever or heel lever and enlarge the corresponding lever. A lengthening of the toe lever will delay heel rise and supports knee extension longer through the stance phase.

In operation of the prosthetic foot 10 of the present invention, the sole plate 12 flexes along its length as a user steps forward. The back portion of the ankle member 14 is rigidly affixed to the heel portion of the sole plate 12 so that the posterior planar portion 22 of the ankle member moves together with the posterior heel portion of the sole plate 12. As the user's weight is transferred anterior or forward following initial contact, the extension portion 26 of the ankle member 14 tilts forward reducing the gap 28 formed between the resilient pad 18 and the extension portion 26. The extension portion 26 then contacts the resilient pad 18 and compresses the resilient pad 18 to arch the sole plate 12 about the resilient pad 18 to reduce and distribute sole plate 12 stresses; thereby promoting a long service life. In addition, the flex of the sole plate 12 improves the dorsiflexion characteristics of the foot allowing it to remain in contact with the ground a longer period of time until full contact with the extension portion 26 is achieved to promote a more natural gait of a user of the prosthetic foot 10. As stated above the resilient pad 18 may be moved anterior in relation to the sole plate 12, as shown in FIG. 2. Movement of the resilient pad 18 in an anterior direction has the effect of increasing the dorsiflexion of the foot, as the gap 28 between the extension portion 26 and the resilient pad 18 has been increased. Similarly, the thickness of the resilient pad 18 may be reduced, as shown in FIG. 4, again to increase the dorsiflexion of the foot. The presence of the resilient pad 18 serves to limit the forward tilt of the ankle assembly 14, and as will be appreciated by those of skill in the art; the degree of motion may be selected by selecting the thickness and/or hardness of the material forming the resilient pad 18.

In an alternative embodiment of the present invention, multiple resilient pads 18 may be positioned between the ankle member 14 and the sole plate 12. For example, a sole plate 12 having a slot formed in the middle of it to allow for passage of a multi piece ankle member may be utilized by the present invention. In such a design a resilient pad 18 may be positioned on opposite sides of the slot between the sole plate 12 and ankle member 14 to perform the same function as outlined above.

The invention has been described in an illustrative manner. It is to be understood that the terminology which has been used is intended to be in the nature of words of description rather than limitation. Many modifications and variations of the invention are possible in light of the above teachings. Therefore, within the scope of the appended claims, the invention may be practiced other than as specifically described.

1. A prosthetic foot comprising:
a sole plate having a flexible body formed of resilient material, the sole plate being elongated along an anterior and posterior axis wherein an anterior portion of the plate defines a toe portion and the posterior portion defines a heel portion;
an ankle member having a planar portion in contact with the sole plate and rigidly affixed to the sole plate at the heel portion thereof, the ankle member having an extension portion positioned anterior of the planar portion and separated from the planar portion by a transition portion;
a resilient pad disposed between the contact portion of the ankle member and the sole plate;
wherein the resilient pad and extension portion of ankle member define a gap such that as a user's weight is transferred anterior following initial contact, the extension portion of the ankle member tilts forward reducing the gap and contacting the resilient pad and arching the sole plate about the resilient pad reducing and distributing sole plate stresses, and wherein the sole plate flexes improving the dorsiflexion of the prosthetic foot for improving the gait of the user.

2. The prosthetic foot of claim 1 wherein the resilient pad is movable in an anterior or posterior direction along the sole plate adjusting the size of the gap defined by the extension portion and the resilient pad adjusting the dorsiflexion of the prosthetic foot.

3. The prosthetic foot of claim 1 wherein a thickness of the resilient pad may be adjusted to change the size of the gap defined by the extension portion and the resilient pad changing the dorsiflexion of the prosthetic foot.

4. The prosthetic foot of claim 1 including a coupler associated with the ankle member, the coupler being operable to join the ankle member to a leg prosthesis.

5. The prosthetic foot of claims 1 wherein the sole plate is formed of a body of composite material including a polymer reinforced with a fibrous material.

6. The prosthetic foot of claim 5 wherein the fibrous material is selected from the group consisting of carbon fibers, glass fibers, ceramic fibers, polymeric fibers, and combinations thereof.

7. The prosthetic foot of claim 6 wherein the sole plate is formed of a glass-reinforced or carbon-reinforced polymer composite, and combinations thereof.

8. The prosthetic foot of claim 7 wherein the sole plate has a flexibility of from 4 to 19 Mpsi.

9. The prosthetic foot of claim 1 wherein the resilient pad has a Shore A hardness in the range of 60-90 Shore A.

10. The prosthetic foot of claim 1 wherein the resilient pad has a density of from 20-60 pounds per cubic foot.

11. The prosthetic foot of claim 1 wherein the resilient pad is formed of a material selected from the group consisting of rubbers, urethane, and closed-cell foams.

12. A prosthetic foot comprising:
a sole plate having a body formed of resilient material, the sole plate being elongated along an anterior and posterior axis wherein an anterior portion of the plate defines a toe portion and the posterior portion defines a heel portion;
an ankle member having a planar portion in contact with the sole plate and rigidly affixed to the sole plate at the heel portion thereof, the ankle member having an extension portion positioned anterior of the planar portion and separated from the planar portion by a transition portion;
a resilient pad disposed between the contact portion of the ankle member and the sole plate;
wherein the sole plate has a flexibility of from 4 to 19 Mpsi for improving the gait of a user.

13. A prosthetic foot comprising:
a sole plate having a body formed of resilient material, the sole plate being elongated along an anterior and posterior axis wherein an anterior portion of the plate defines a toe portion and the posterior portion defines a heel portion;
an ankle member having a planar portion in contact with the sole plate and rigidly affixed to the sole plate at the heel portion thereof, the ankle member having an extension portion positioned anterior of the planar portion and separated from the planar portion by a transition portion;
a resilient pad disposed between the contact portion of the ankle member and the sole plate;
wherein the resilient pad is moveable anterior and posterior to adjust the dorsiflexion of the prosthetic foot.
